(19) United States
(12) Patent Application Publication
(10) Pub. No.: US 2008/0098816 A1
(43) Pub. Date: May 1, 2008

(54) ULTRASONIC PROBE AND ULTRASONIC DIAGNOSTIC APPARATUS

(76) Inventors: Yohachi Yamashita, Yokohama-shi (JP); Yasuharu Hosono, Yokohama-shi (JP)

Correspondence Address: OBLON, SPIVAK, MCCLELLAND MAIER & NEUSTADT, P.C., 1940 DUKE STREET, ALEXANDRIA, VA, 22314, US

(21) Appl. No.: 11/682,589

(22) Filed: Mar. 6, 2007

(30) Foreign Application Priority Data
Oct. 31, 2006 (JP) 2006-297115

(51) Int. Cl. G01N 29/00; A61B 8/00

(52) U.S. Cl. 73/596; 310/327; 310/335

(57) ABSTRACT

An ultrasonic probe comprises an acoustic backing layer made of a composite resin material including a resin and a plurality of bonded fibers contained the resin, each of the bonded fibers being formed of a plurality of zinc oxide fibers bonded to each other at one edge portions and extending in different directions in the other edge portions, the composite resin material exhibiting an acoustic impedance of 1.3 to 6 MRayls at 25° C., a plurality of channels arranged on the acoustic backing layer with space, each channel having a piezoelectric element and an acoustic matching layer formed on the piezoelectric element, and an acoustic lens formed to cover at least the surface of the acoustic matching layer of each channel.

### CROSS-REFERENCE TO RELATED APPLICATIONS

This application is based upon and claims the benefit of priority from prior Japanese Patent Application No. 2006-297115, filed Oct. 31, 2006, the entire contents of which are incorporated herein by reference.

### BACKGROUND OF THE INVENTION

1. Field of the Invention

The present invention relates to an ultrasonic probe for transmitting ultrasonic signals to, for example, an object and for receiving the ultrasonic signals reflected from the object and also relates to an ultrasonic diagnostic apparatus comprising the ultrasonic probe.

2. Description of the Related Art

An array type ultrasonic probe performing the function of transmitting and receiving ultrasonic signals is used mainly in a medical ultrasonic diagnostic apparatus or an ultrasonic image inspecting apparatus, which transmits an ultrasonic signal to an object and receives the reflected signal (echo signal) coming from within the object for forming an image of the object. The array-type ultrasonic probe is known to include a one dimensional array-type ultrasonic probe in which 50 to 300 channels each including a piezoelectric element and an acoustic matching layers arranged on the piezoelectric element are arranged in one dimensional direction, and a two dimensional array-type ultrasonic probe in which 500 to 10,000 channels noted above are arranged in a two dimensional direction.

The ultrasonic probe is operated for performing diagnosis under the state that the ultrasonic probe on the side of, for example, the acoustic lens contacts the object so as to permit the ultrasonic signals to be transmitted from the front surface of the piezoelectric element into the object. The ultrasonic signals are converted on a prescribed position within the object by the electronic focus depending on the drive timing of the piezoelectric element and by the focus of the acoustic lens. Since the ultrasonic signal can be transmitted in a prescribed range within the object by controlling the drive timing of the piezoelectric element, it is possible to obtain an ultrasonic image (tomographic image) within the prescribed range by receiving and processing the echo signals coming from the object. It should be noted that the ultrasonic signal can also be released to the back side of the channel by the driving of the piezoelectric element. Therefore, the ultrasonic probe is designed to permit the ultrasonic signal released to the back side of the channel to be absorbed by the acoustic backing layer arranged on the back side of the channel so as to prevent the ultrasonic reflection. In particular, in the two dimensional array-type ultrasonic probe in which channels having small areas of 0.04 to 0.3 mm² are arranged in a two dimensional direction, the acoustic backing layer is required to be formed of a material having properties such as strength and uniformity, which are severer than those of the material of the one dimensional ultrasonic probe.

A material used for manufacturing the acoustic backing layer is disclosed in, for example, “Martha G. Grewe et al., IEEE transaction Ultrasonic Ferroelectrics and Frequency control, vol. 37, No. 6, p. 506, 1990”. It is disclosed that a powdery material having a high density, such as tungsten (W), alumina (Al₂O₃), zinc oxide (ZnO), or iron oxide (Fe₂O₃), is mixed with an epoxy resin used as a base resin so as to prepare a material having a density of about 2.0 (g/cm³), an acoustic velocity of about 2500 m/s, and an acoustic impedance of about 5 MRayls. The material thus prepared is used for manufacturing an acoustic backing layer. It is also taught in the literature quoted above that a powdery material having a high density, such as W metal, ZnO, or Fe₂O₃ is mixed with a rubber-based material such as chloroprene rubber (CR), isoprene rubber (IR) or an urethane rubber (AU), which is used as the base resin, so as to prepare a material having a density of about 3.0 (g/cm³), an acoustic velocity of about 1,500 m/s, and an acoustic impedance of about 5 MRayls. The material thus prepared is also used for manufacturing an acoustic backing layer.

JP-A 7-233278 (KOKAI) teaches that a resin molding, which contains tungsten, is used in an acoustic backing layer. The resin molding is obtained by molding a powdery composition obtained by using W metal and a thermosetting resin as main materials.

JP-A 9-127955 (KOKAI) discloses an acoustic backing layer prepared by using a preform and a matrix material, and teaches that the preform has a linear fibrous texture, a planar fibrous texture or a three dimensional fibrous texture.

JP-A 2006-33801 (KOKAI) discloses an acoustic backing layer prepared by adding a filler selected from the group consisting of a carbon fiber, a silicon carbide fiber, and an alumina fiber and a powdery material such as zinc oxide, zirconium oxide, aluminum oxide or aluminum nitride are added to an ethylene-vinyl acetate copolymer containing 20 to 80% by weight of vinyl acetate units.

On the other hand, Japanese Patent No. 2598829 discloses an electric wave absorbing body prepared by mixing short semiconductive fibers having an aspect ratio of 10 to 10,000, a diameter of 0.1 to 100 μm and a resistivity of 10⁻² to 10⁵ Ω with a part of tetrapod-like zinc oxide whisker.

However, the acoustic backing layer disclosed in any of JP-A 7-233278 (KOKAI), JP-A 9-127955 (KKAI), JP-A 2006-33801 (KOKAI) and “Martha G. Grewe et al., IEEE transaction Ultrasonic Ferroelectrics and Frequency control, vol. 37, No. 6, p-506, 1990”, which are pointed out above, gives rise to problems as pointed out in the following. Specifically, in order to improve the quality of the tomographic image formed by the ultrasonic diagnostic apparatus and to improve the sensitivity of the ultrasonic probe, it is necessary to decrease the number of defective channels and to decrease the nonuniformity among the channels. The problem relating to the defective channel is particularly serious when it comes to a two dimensional array probe having a small channel area not larger than 0.1 mm². The cause of the defective channel is considered to be as follows. To be more specific, a mesa structure is formed between the trenches formed on one main surface of the acoustic backing layer. It is possible for this mesa structure to have an insufficient mechanical strength. As a result, it is possible for the piezoelectric element of the channel formed on the mesa structure to collapse, together with the mesa structure, so as to make it impossible to use the channel. It should be noted that, where particles of W metal or ferrite sized at several microns to scores of microns are contained as a filler in the acoustic backing layer in an amount of 10 to 100% by weight, the resin or rubber forming the acoustic backing layer is rendered brittle. It follows that breakage and peeling are generated between the rubber and the filler due to the stress in the stage of cutting with, for example, a blade the ultrasonic probe from the acoustic matching layer toward the acoustic backing layer through the piezoelectric element. In other words, if the size of the piezoelectric element is diminished, the size of the acoustic backing layer is also diminished. As a result, the acoustic backing layer is folded or the acoustic backing layer and the piezoelectric element peel away from each other due to the stress that is generated in the stage of cutting the acoustic backing layer that includes the region in the vicinity of bonding surface of the piezoelectric element at a pitch of 50 to 100 μm. Particularly, difficulty is generated in the case where the acoustic backing layer contains powdery particles alone, e.g., W particles alone, as described in JP-A 7-233278 (KOKOAI), referred to above. Also, where a large amount of fibers, such as carbon fibers or potassium titanate, are contained in a large amount in a resin as disclosed in JP-A 9-127955 (KOKAI) or JP-A 2006-33801 (KOKAI), the electric conductivity of the fiber makes it difficult to ensure a sufficient insulation between the adjacent channels, which gives rise to an inconvenience that a pulse current cannot be applied. For overcoming the difficulty, the space between the adjacent channels is loaded with an insulating material used as a filler such as silicone rubber. However, the difference in the amount of the filler causes the acoustic impedances of the backing materials to differ from each other among fine channels (0.02 to 0.3 mm²) used in a two dimensional array. It follows that a nonuniformity tends to be generated in the sensitivity of transmitting and receiving the ultrasonic signals.

In the electric wave absorber disclosed in Japanese Patent No. 2598829, the entire resin is made electrically conductive so that it can absorb the electric wave. This electric absorber is used for absorbing the transverse wave, which consists of an electric field and a magnetic field in the GHz band. On the other hand, the backing layer used in an ultrasonic probe is intended to absorb an ultrasonic wave that consists of a longitudinal wave composed of compressional waves having a frequency of 1 to 10 MHz and, thus, differs from the electric wave absorber in the application, too.

Recent ultrasonic probes of the two dimensional array-type sometimes include several thousand piezoelectric elements, each sized at 200 μm×200 μm, and having a central frequency of about 3 MHz. Therefore, it is very important to improve the mechanical strength and the uniformity of the acoustic backing layer. In this respect, each of JP-A 7-233278 (KOKAI) and JP-A 9-127955 (KOKAI) referred to above teaches the idea of mixing a fibrous material as a preform with a matrix material. However, the strength and the uniformity in a small area of the backing layer are not considered at all in each of these patent documents.

Also, in order to avoid a temperature elevation on the surface of the ultrasonic probe (surface of the acoustic lens), it is desirable to use a small and light backing layer excellent in thermal conductivity. In order to obtain a small and light backing layer, it is important to develop a material having a large ultrasonic wave acoustic attenuation factor, and a low density. Such being the situation, it is desirable to use a material having an acoustic impedance of 1.5 to 6 MRayls. Further, it is important for the backing layer to exhibit insulating properties.

### BRIEF SUMMARY OF THE INVENTION

According to the present invention, there is provided an ultrasonic diagnostic apparatus, comprising:

an ultrasonic probe comprising an acoustic backing layer made of a composite a resin material including resin and a plurality of bonded fibers contained in the resin, each of the bonded fibers being formed of a plurality of zinc oxide fibers bonded to each other at one edge portions and extending in different directions in the other edge portions, the composite resin material exhibiting an acoustic impedance of 1.3 to 6 MRayls at 25° C., a plurality of channels arranged on the acoustic backing layer with space, each channel having a piezoelectric element and an acoustic matching layer formed on the piezoelectric element, and an acoustic lens formed to cover at least the surface of the acoustic matching layer of each channel; and

an ultrasonic diagnostic apparatus connected to the ultrasonic probe via a cable.

### BRIEF DESCRIPTION OF THE SEVERAL VIEWS OF THE DRAWING

FIG. 1 is an oblique view (partly broken away) showing the construction of a one dimensional array-type ultrasonic probe according to an embodiment;

FIG. 2 is an oblique view showing the construction of the gist portion of the ultrasonic probe shown in FIG. 1;

FIG. 3 is a cross-sectional view along the line III-III shown in FIG. 2;

FIG. 4 is a cross-sectional view exemplifying the construction of the acoustic backing layer used in the embodiment;

FIG. 5 is a cross-sectional view exemplifying another construction of the acoustic backing layer used in the embodiment;

FIG. 6 shows the construction of the fibers contained in the composite resin material constituting the acoustic backing layer used in the embodiment;

FIG. 7 is an oblique view showing the construction in the gist portion of the two dimensional array-type ultrasonic probe according to an embodiment;

FIG. 8 is a partial cross-sectional view along the line VIII-VIII shown in FIG. 7;

FIG. 9 is a partial cross-sectional view along the line IX-IX shown in FIG. 7; and

FIG. 10 schematically shows the construction of an ultrasonic diagnostic apparatus according to an embodiment.

### DETAILED DESCRIPTION OF THE INVENTION

An ultrasonic probe and an ultrasonic diagnostic apparatus according to embodiments of the present invention will now be described in detail.

The ultrasonic probe according to the embodiment comprises an acoustic backing layer. A plurality of channels are arranged on the acoustic backing layer with space. Each channel includes a piezoelectric element and an acoustic matching layer formed on the piezoelectric element. The piezoelectric element comprises a piezoelectric body made of, for example, a lead zirconium titanate (PZT) based piezoelectric ceramic material or a relaxor based single crystal material and first and second electrodes formed on both surfaces of the piezoelectric body. Each of the first and second electrodes is made of, for example, a Ag—Pd alloy. The acoustic matching layer is used in the form of a single layer structure or a laminate structure including a plurality of layers. A mixed material prepared by adding a filler such as a fiber to, for example, a polyurethane rubber, polyethylene, a silicone rubber or an epoxy resin is used for preparing the acoustic matching layer. The acoustic lens is formed to cover the surface of the acoustic matching layer of each channel. The acoustic lens is formed of a mixed material prepared by adding an inorganic filler to, for example, a silicone rubber. The mixed material noted above exhibits an acoustic impedance of 1.3 to 1.7 MRayls at 25° C.

The acoustic backing layer is made of a composite resin material including resin and a plurality of bonded fibers contained in the resin. Each of the bonded fibers is formed of a plurality of zinc oxide fibers bonded to each other at one edge portions and extending in different directions in the other edge portions. The composite resin material exhibits an acoustic impedance of 1.3 to 6 MRayls at room temperature of 25° C.

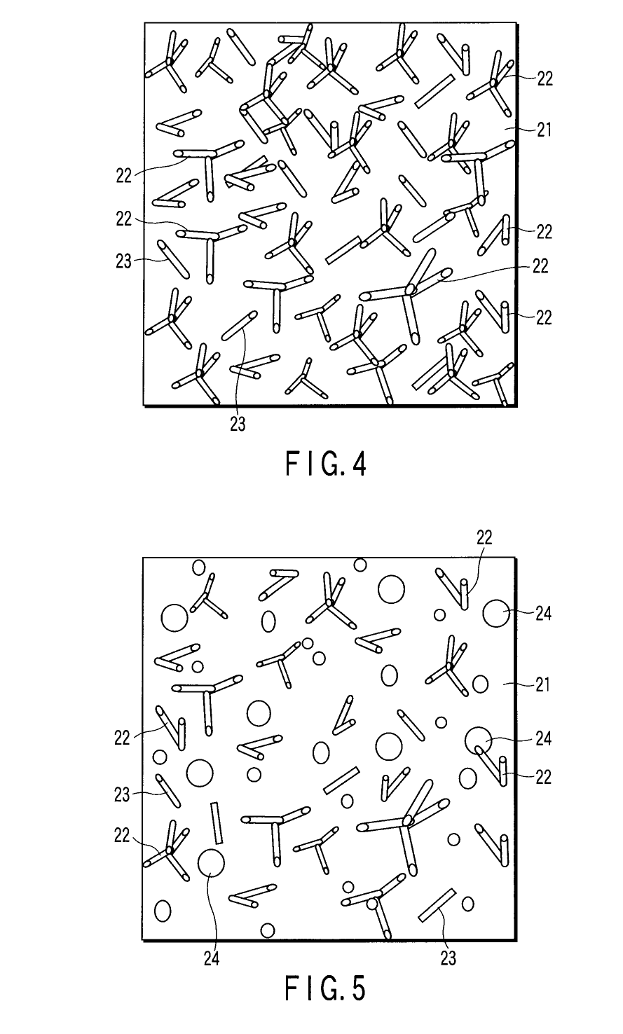

In the acoustic backing layer made of the particular composite resin material described above, the bonded fibers 22 each formed of a plurality of zinc oxide fibers bonded to each other in one edge portions and extending in different directions in the other edge portions are dispersed in the resin 21, as shown in, for example, FIG. 4. Incidentally, a reference numeral 23 shown in FIG. 4 denotes a piece of fiber broken from the bonded fiber 22 in the process of manufacturing the acoustic backing layer from the composite resin material. It is acceptable for the amount of the fiber pieces 23 to be not larger than 30% by volume based on the amount of the bonded fibers 22. If the amount of the fiber pieces 23 exceeds 30% by volume, the acoustic attenuation factor tends to be lowered.

The resin includes, for example, an epoxy resin, a silicone rubber, a nitrile based rubber, an isoprene based rubber and chloroprene rubber, a butyl rubber, a urethane rubber, an ethylene-propylene-diene (EPDM) rubber, a fluorosilicone rubber, and a fluorinated elastomer (e.g., “Siful” resin manufactured by Shin-Etsu Chemical Co., Ltd.). These resins can be used singly or in the form of a mixture. Particularly, it is desirable to use an epoxy resin as the resin contained in the composite resin material. It is desirable for the epoxy resin to be obtained by curing, for example, bisphenol A type or F type with a modified aliphatic polyamine, an anhydride or polythiol.

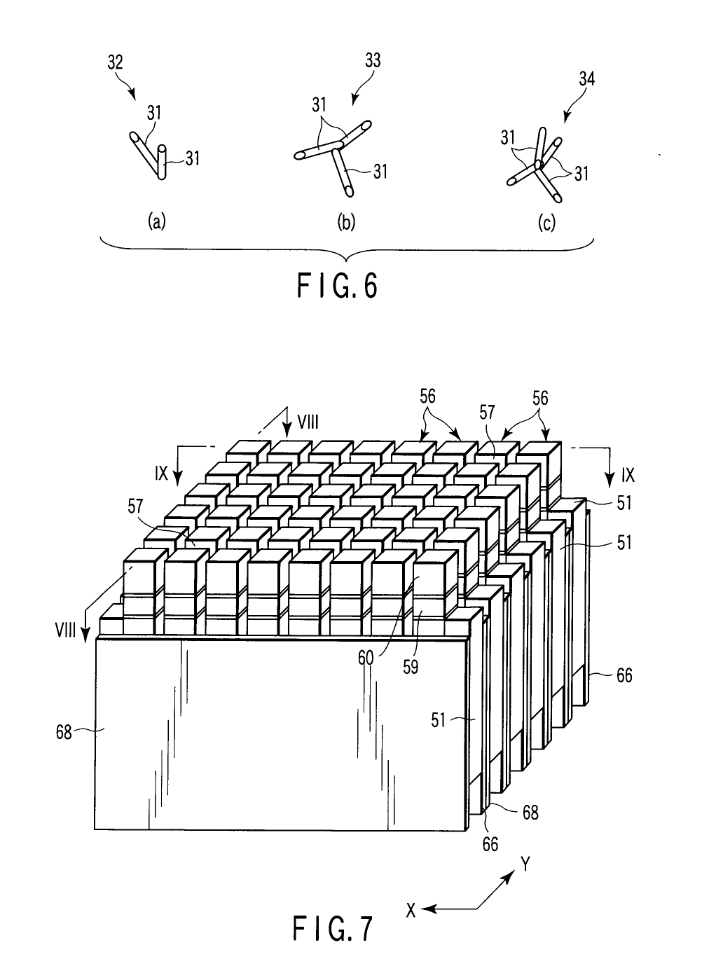

The bonded fiber includes, for example, a bonded fiber 32 consisting of two zinc oxide fibers 31 bonded to each other at one edge portions and extending in different directions in the other edge portions, as shown in FIG. 6 (a), a bonded fiber 33 consisting of three zinc oxide fibers 31 bonded to each other at one edge portions and extending in different directions in the other edge portions, as shown in FIG. 6 (b), and a bonded fiber 34 consisting of four zinc oxide fibers 31 bonded to each other at one edge portions and extending in different directions in the other edge portions, as shown in FIG. 6 (c). Of these bonded fibers, it is possible for the zinc oxide fibers extending in different directions to be equal to or different from each other in length. Among these bonded fibers, it is particularly desirable to use a tetrapod-shaped bonded fiber consisting of four zinc oxide fibers bonded to each other at one edge portions and extending in different directions in the other edge portions. The tetrapod-shaped bonded fiber can be manufactured by subjecting, for example, a metal zinc powder to a heat treatment at 800 to 1050° C. for 1 to 30 minutes under an atmosphere containing oxygen in a concentration not higher than several percent. Also, it is possible for the resin to be loaded with bonded fibers including the same number of zinc oxide fibers extending in different directions or to be loaded with a mixture of at least two kinds of the bonded fibers selected from the bonded fibers shown in FIGS. 6(a) to 6(c).

It is desirable for the zinc oxide fiber constituting the bonded fiber to have a diameter not larger than 10 μm, preferably 1 to 10 μm, and a length at least 5 times as much as the diameter. It is desirable for the bonded fibers to be contained and dispersed in the resin in an amount of 3 to 20% by volume based on the total amount of the resin and the bonded fibers.

If the diameter of the zinc oxide fiber exceeds 10 μm, the ultrasonic wave transmitted from the piezoelectric element of the channel tends to be reflected easily, and the zinc oxide fiber fails to improve sufficiently the effect of improving the strength of the acoustic backing layer in the stage of cutting the laminate structure including the acoustic backing layer. In addition, the nonuniformity in the sensitivity of the ultrasonic probe tends to be increased when the channel is formed to have a fine area (e.g., 0.04 mm² to 0.3 mm²). Also, if the length of the zinc oxide fiber is smaller than 5 times the diameter, it is difficult to improve sufficiently the effect of improving the strength of the acoustic backing layer formed of the composite resin material, as well as the effects on the thermal conductivity, high acoustic attenuation factor, reduction of the acoustic velocity and the reduction of the nonuniformity of the acoustic backing layer formed of the composite resin material. Particularly, if the length of the zinc oxide fiber exceeds 50 μm, it is difficult to maintain the desired shape of the bonded fiber.

If the content of the bonded fibers in the composite resin material is lower than 3% by volume, it is difficult to improve sufficiently the mechanical strength the acoustic backing layer formed of the composite resin material and to improve the effects on the heat dissipating properties, high acoustic attenuation factor, reduction of the acoustic velocity and the reduction of the nonuniformity of the acoustic backing layer formed of the composite resin material. On the other hand, if the content of the bonded fibers in the composite resin material exceeds 20% by volume, it is difficult to load the bonded fibers in the resin. It is more desirable for the bonded fibers to be contained in the resin in an amount of 5 to 15% by volume.

It is acceptable for the composite resin material to further contain particles having, for example, an average particle diameter of 2 to 15 μm and made of, for example, metal tungsten, tungsten oxide, metallic tantalum, metallic iron or iron oxide. It is desirable for the particles to be contained in the resin in an amount not larger than 10% by volume, preferably in an amount of 1 to 5% by volume, based on the sum of the resin and the particles. The acoustic backing layer made of the composite resin material prepared by allowing a resin to contain the particles together with the bonded fibers exhibits a high strength, high insulating properties, and a high thermal conductivity while maintaining a low value of the acoustic impedance. Also, where the channel is formed to have a fine area (e.g., not larger than 0.3 mm² and falling within a range of 0.04 to 0.3 mm²), the acoustic backing layer exhibits a uniform sensitivity and a large acoustic attenuation factor. It is desirable for the bonded fibers and the particles to be contained in the resin in a volume ratio of 3:0.1 to 2:1.

The acoustic backing layer formed of the composite resin material described above is constructed as shown in, for example, FIG. 5. As shown in the drawing, the bonded fibers 22 each consisting of a plurality of zinc oxide fibers bonded to each other at one edge portions and extending in different directions in the other edge portions are dispersed in the resin 21 together with the particles 24 having an average particle diameter of 2 to 15 μm. Incidentally, the reference numeral 23 shown in FIG. 5 denotes a piece of fiber broken from the bonded fiber 22 in the process of manufacturing the acoustic backing layer from the composite resin material. It is acceptable for the fiber pieces 23 to be contained in an amount not larger than 30% by volume based on the amount of the bonded fibers contained in the resin 21.

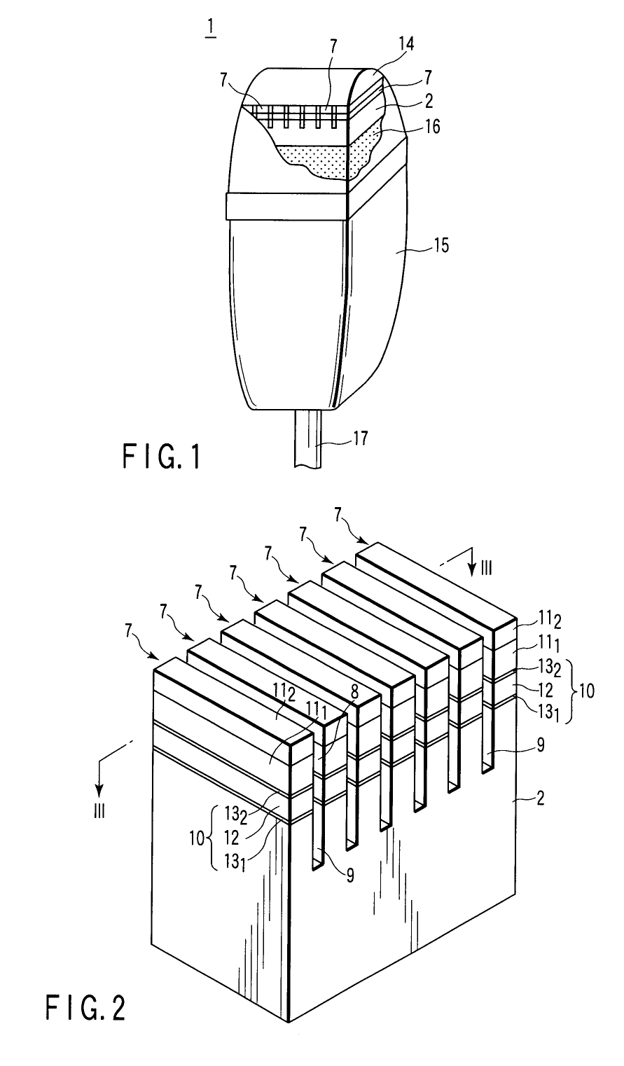

The ultrasonic probe, e.g., a one dimensional array-type ultrasonic probe, according to the embodiment will now be described with reference to FIGS. 1 to 3.

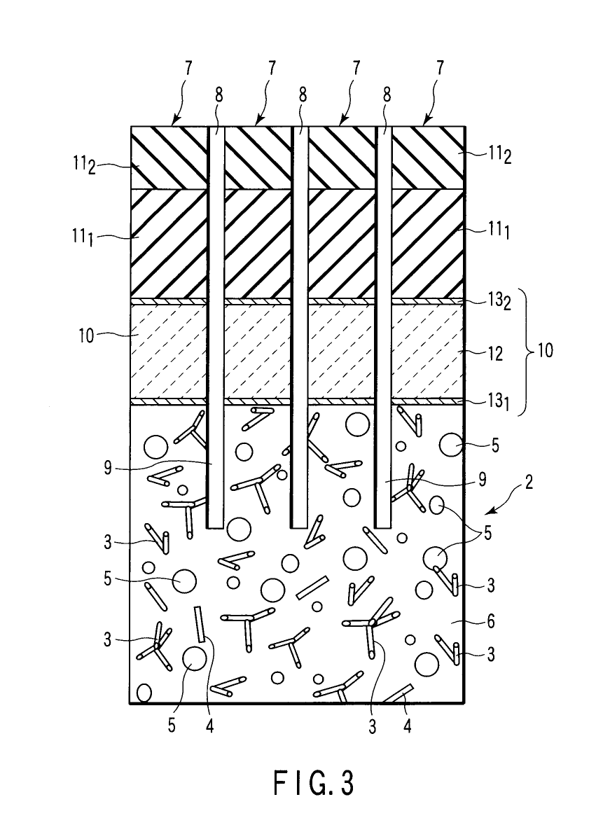

FIG. 1 is an oblique view, partly broken away, showing the construction of a one dimensional array-type ultrasonic probe according to the embodiment, FIG. 2 is an oblique view showing the gist portion of the ultrasonic probe shown in FIG. 1, and FIG. 3 is a cross-sectional view along the line III-III shown in FIG. 2.

As shown in the drawings, the one dimensional array-type ultrasonic probe 1 comprises an acoustic backing layer 2. As shown in FIG. 3, the acoustic backing layer 2 is made of a composite resin material prepared by allowing a plurality of bonded fibers 3 to be contained in a resin 6. Each of the bonded fibers 3 is formed of a plurality of zinc oxide fibers bonded to each other at one edge portions and extending in different directions in the other edge portions. A plurality of fiber pieces 4 broken from the bonded fibers 3 and a plurality of particles 5 are also contained in the resin 6. The composite resin material of the particular construction exhibits an acoustic impedance of 1.3 to 6 MRayls at room temperature of 25° C.

A plurality of channels 7 are arranged on the acoustic backing layer 2 with space 8. Trenches 9 communicating with the spaces 8 are formed in the upper portion of the acoustic backing layer 2. Incidentally, it is possible to load a relatively soft resin exhibiting a low acoustic impedance and a high acoustic attenuation factor such as a silicone resin in the space 8 between the adjacent channels 7 so as to maintain a required mechanical strength of the ultrasonic probe.

Each channel 7 includes a piezoelectric element 10 and an acoustic matching layer of at least a single layer structure formed on the piezoelectric element 10. In the example shown in FIGS. 2 and 3, each channel 7 includes two acoustic matching layers, consisting of a first acoustic matching layer 11₁ and a second acoustic matching layer 11₂. As shown in the drawings, the piezoelectric element 10 comprises a piezoelectric body 12 made of, for example, a lead zirconium titanate (PZT) based piezoelectric ceramic material or a relaxor based single crystal material and first and second electrodes 13₁, 13₂ formed on both surfaces of the piezoelectric body 12. The first electrode 13₁ of the piezoelectric element 10 is bonded and fixed to the acoustic backing layer 2 via, for example, an epoxy resin based adhesive layer (not shown). The first acoustic matching layer 11₁ is bonded and fixed to the second electrode 13₂ of the piezoelectric element 10 via, for example, an epoxy resin based adhesive layer (not shown). Further, the second acoustic matching layer 11₂ is bonded and fixed to the first acoustic matching layer 11₁ via, for example, a rubber based adhesive layer (not shown).

Further, an acoustic lens 14 is formed on the surface of the second acoustic matching layer 11₂ of the channel 7.

The acoustic backing layer 2, the plural channels 7 and the acoustic lens 14 are housed in a case 15 having an open upper edge such that the backing layer 2 is disposed on a support table 16. Housed in the case 15 are a control circuit for controlling the drive timing of the piezoelectric element for each channel 7 and a signal processing circuit (not shown) including an amplifying circuit for amplifying the signal received by the piezoelectric element 10. One edge of the signal side printed wiring board and one edge of the ground side printed wiring board are connected respectively to the first and second electrodes 13₁, 13₂ of the piezoelectric element 10, and the other edges of the wiring boards noted above are connected to the control circuit and the signal processing circuit, respectively, which are housed in the case 15. A cable 17 is inserted into the case 15 through the edge portion opposite to the acoustic lens 14 such that the tip of the cable 17 is connected to the signal processing circuit (not shown).

In the array-type ultrasonic probe of the construction described above, a voltage is applied between the first and second electrodes 13₁ and 13₂ of the piezoelectric element 10 for each channel 7 so as to bring about resonance of the piezoelectric body 12. As a result, an ultrasonic wave is radiated (transmitted) through the acoustic matching layer (first and second acoustic matching layers 11₁, 11₂) and the acoustic lens 14. When echo signals are received, the piezoelectric body 12 of the piezoelectric element 10 included in each channel 7 is vibrated by the ultrasonic wave received through the acoustic lens and the acoustic matching layer (first and second acoustic matching layers 11₁, 11₂) included in each channel 7. The vibration is converted into electric signals so as to obtain an image.

How to manufacture the one dimensional array-type ultrasonic probe according to the embodiment will now be described.

In the first step, an acoustic backing layer is manufactured by using a composite resin material exhibiting an acoustic impedance of 1.3 to 6 MRayls at room temperature of 25° C. The composite resin material is prepared with a resin and a plurality of bonded fibers contained in the resin. Each of the bonded fibers consists of a plurality of zinc oxide fibers bonded to each other at one edge portions and extending in different directions in the other edge portions. To be more specific, the acoustic backing layer is manufactured by dispersing a plurality of bonded fibers in a liquid resin, such as a liquid epoxy resin, followed by molding the liquid resin. In the case of using a liquid resin as pointed out above, it is possible to suppress the breakage of the plural bonded fibers in the dispersing process of the bonded fibers.

In the next step, the piezoelectric element and the first and second acoustic matching layers are laminated one upon the other on the acoustic backing layer with an epoxy resin based adhesive of a low viscosity interposed between the laminated members. Then, the laminate structure thus obtained is baked at, for example, 120° C. for about one hour so as to cure each of the epoxy resin based adhesives, with the result that the piezoelectric element is bonded and fixed to the acoustic backing layer, the first acoustic matching layer is bonded and fixed to the piezoelectric element, and the second acoustic matching layer is bonded and fixed to the first acoustic matching layer.

In the next step, a dicing (cutting) process is applied to the laminate structure, i.e., applied from the second acoustic matching layer toward the acoustic backing layer, at a width (pitch) of, for example, 50 to 200 μm so as to divide the laminate structure into a plurality of sections forming a one dimensional array, thereby forming a plurality of channels each including the piezoelectric element, and first and second acoustic matching layers. In this stage, trenches corresponding to the spaces between the adjacent channels are formed in the surface region of acoustic backing layer. In the next step, the spaces between the adjacent channels are loaded as required with a relatively soft resin having, for example, a low acoustic impedance and a high acoustic attenuation factor, such as a silicone rubber, so as to maintain a required mechanical strength of each channel. Subsequently, an acoustic lens is bonded and fixed to the second acoustic matching layer of each channel by using a silicone rubber based adhesive layer. Then, the acoustic backing layer, the plural channels and the acoustic lens are housed in a case. Further, a control circuit and a signal processing circuit housed in the case are connected to the electrodes of the piezoelectric element, and a cable is connected to the signal processing circuit so as to manufacture a one dimensional array-type ultrasonic probe.

A two dimensional array-type ultrasonic probe according to an embodiment will now be described with reference to FIGS. 7 to 9.

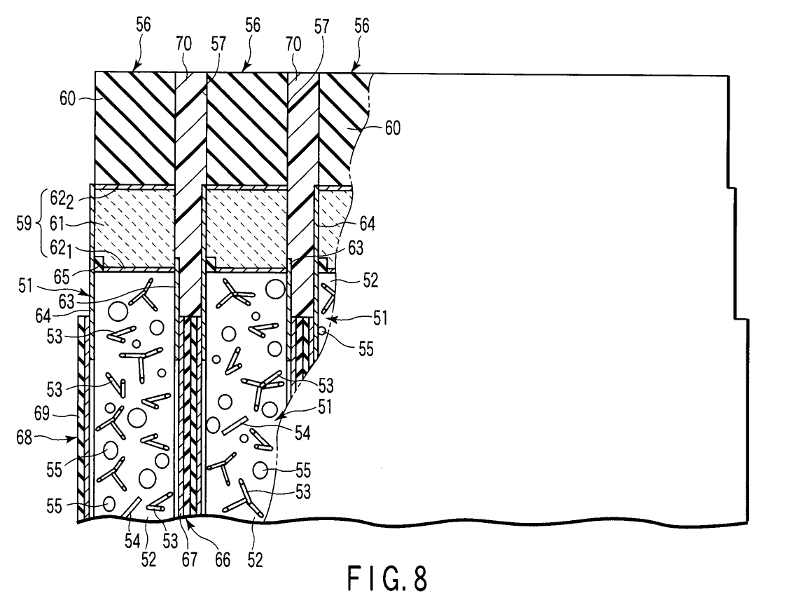

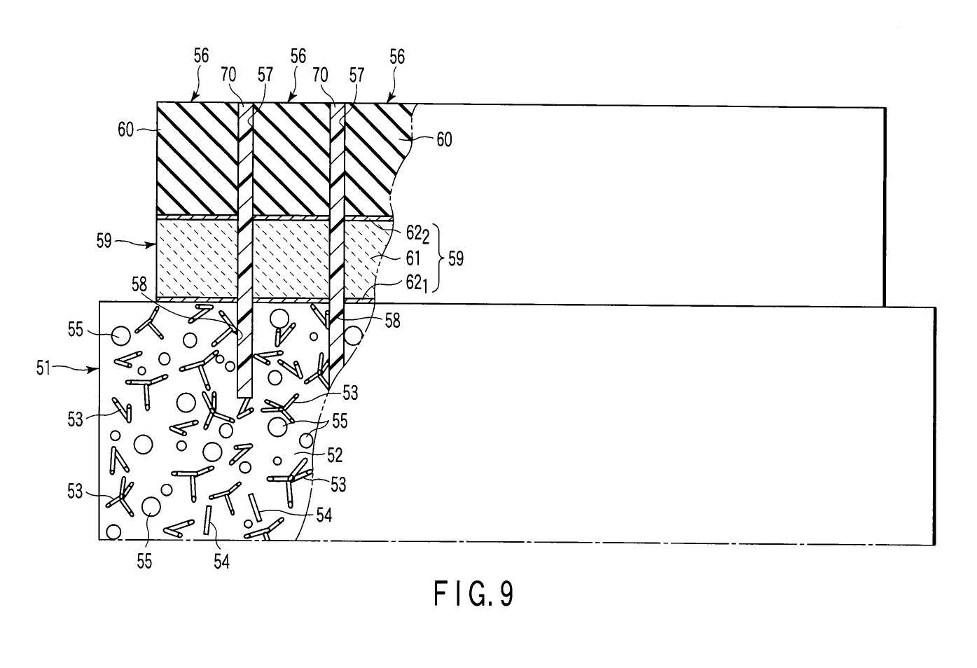

FIG. 7 is an oblique view showing the construction in the gist portion of the two dimensional array-type ultrasonic probe according to the embodiment, FIG. 8 is a partial cross-sectional view along the line VIII-VIII shown in FIG. 7, and FIG. 9 is a partial cross-sectional view along the line IX-IX shown in FIG. 7.

As shown in FIG. 9, the acoustic backing layer 51 is made of a composite resin material having an acoustic impedance of 1.3 to 6 MRayls at room temperature of 25° C. The composite resin material is prepared by allowing a plurality of bonded fibers 53 to be contained in a resin 52. Each of the bonded fibers is formed of a plurality of zinc oxide fibers bonded to each other at one edge portions and extending in different directions at the other edge portions. The resin 52 also contains a plurality of fiber pieces 54 broken from the bonded fibers 53 and a plurality of particles 55 having an average particle diameter of 2 to 15 μm.

The plural channels 56 are arranged in two dimensional directions (X and Y directions) in a manner to form a matrix on the acoustic backing layer 51. As shown in FIG. 8, the acoustic backing layer 51 is separated into a plurality sections in the Y-direction. To be more specific, the plural channels 56 are arranged on the common backing layer 51 in the X-direction as shown in FIG. 9, and arranged on separated backing layers 51 in the Y-direction, as shown in FIG. 8. Also, trenches 58 are formed in the acoustic backing layer 51 in a manner to communicate with the spaces 57 between the adjacent channels 56 arranged in the X-direction, as shown in FIG. 9.

Each channel 56 includes a piezoelectric element 59 and an acoustic matching layer 60 of, for example, a single layer structure, which is arranged on the piezoelectric element 59. Incidentally, it is possible for the acoustic matching layer 60 to be in the form of a laminate structure prepared by laminating a plurality of layers one upon the other.

The piezoelectric element 59 is arranged on the acoustic backing layer 51 and comprises a piezoelectric body 61 made of, for example, a lead zirconium titanate (PZT) based piezoelectric ceramic material or a relaxor type single crystal material and first and second electrodes 62₁ and 62₂ made on the both surfaces of the piezoelectric body 61. Each of the first and second electrodes 62₁ and 62₂ is formed of, for example, a Ag—Pd alloy.

As shown in FIG. 8, a signal side electrode 63 is formed to extend from one side surface of the piezoelectric element 59 so as to reach the acoustic backing layer 51 and is connected to the first electrode 62₁ exposed on one side surface of the piezoelectric element 59. On the other hand, a ground side electrode 64 is formed to extend from another side surface of the piezoelectric element 59 so as to reach the acoustic backing layer 51 and is connected to the second electrode 62₂ exposed on the other side surface of the piezoelectric element 59. Incidentally, the first electrode 62₁ exposed on the other side surface is partly cut away (or recessed), and an insulating member 65 consisting of, for example, an epoxy resin is buried in the recessed portion. As a result, the insulating member 65 is interposed between the ground side electrode 64 and the first electrode 62₁ when the ground side electrode 64 extends from the second electrode 62₂ exposed on the other surface of the piezoelectric element 59 across the portion of the first electrode 62₁ so as to reach the acoustic backing layer 51. It follows that the ground electrode 64 and the first electrode 62₁ are prevented from being electrically connected to each other.

A signal side printed wiring board (e.g., a flexible printed wiring board 66 for signals) comprises signal lines 67 patterned on the surface at the arranging pitch of the channels 56. These signal lines 67 are electrically connected to the signal side electrodes 63 at the positions of the acoustic backing layer 51. On the other hand, the ground side printed wiring board (e.g., a flexible printed wiring board 68 for the ground) includes ground wires 69 patterned on the surface at the arranging pitch of the channels 56. These ground wires 69 are electrically connected to the ground side electrode 64 at the position of the acoustic backing layer 51. The connection between the signal side printed wiring board 66 and the signal side electrode 63 and the connection between the ground side printed wiring board 68 and the ground side electrode 64 are carried out in the spaces extending in the Y-direction between the adjacent backing layers 51, as shown in FIG. 8. Incidentally, a common ground wire can be obtained by using a ground electrode plate that is not patterned.

A filler member 70 is formed in each of the space 57 formed between the adjacent channels 56 arranged in the X-direction, in the trench 58 of the acoustic backing layer 51 communicating with the space 57, in the space 57 between the adjacent channels 56 arranged in the Y-direction, and in the space between the adjacent backing layers 51.

An acoustic lens (not shown) is formed on the acoustic matching layer 60 of each of a plurality of channels 56.

The acoustic backing layer 51, the plural channels 56 and the acoustic lens (not shown) are housed in a case (not shown). Also housed in the case are a control circuit for controlling the drive timing of the piezoelectric element 59 of each channel and a signal processing circuit (not shown) including an amplifying circuit for amplifying the signal received by the piezoelectric element 59. A signal line 67 and a ground line 69 of the flexible printed wiring boards 66 and 68 are electrically connected to these circuits.

The signal side and ground side printed wiring boards 66 and 68 are not limited to the flexible printed wiring boards. It is also possible for the flexible printed wiring boards 66 and 68 to be replaced by rigid printed wiring boards each prepared by forming at least one kind of a conductive layer (signal line, ground line) selected from the group consisting of Au, Cr, Cu and Ni on the surface of a substrate formed of a composite material prepared by weaving glass unwoven fabric in an epoxy resin.

How to manufacture the two dimensional array-type ultrasonic probe described above will now be described in the following as an example.

In the first step, a plate-like acoustic backing layer is made of a composite resin material exhibiting an acoustic impedance of 1.3 to 6 MRayls at room temperature of 25° C. and prepared by allowing a plurality of bonded fibers to be contained in a resin. Each of bonded fibers consists of a plurality of zinc oxide fibers bonded to each other at one edge portions and extending in different directions in the other edge portions. To be more specific, a plate-like acoustic backing layer is obtained by adding a plurality of bonded fibers to a liquid resin, such as a liquid epoxy resin, and dispersing the bonded fibers in the liquid resin, followed by molding the resin composition. By using a liquid resin as described above, it is possible to prevent the plural bonded fibers from being broken in the dispersing stage.

In the next step, a plate-like piezoelectric element is bonded to the plate-like acoustic backing layer with an epoxy adhesive. Then, a plate-like laminate structure comprising a plate-like acoustic backing layer, a plate-like piezoelectric element, and a plate-like acoustic matching layer is obtained by bonding, for example, a plate-like acoustic matching layer to the plate-like piezoelectric element by using an epoxy adhesive. Further, the plate-like laminate structure is cut at a width of, for example, about 400 μm by using a dicing saw so as to obtain a plurality of sliver laminate structures each including a sliver piezoelectric element and a sliver acoustic matching layer, which are laminated on the sliver acoustic backing layer in the order mentioned.

In the next step, a signal side electrode and a ground side electrode are formed by, for example, a sputtering method on both side surfaces of the sliver piezoelectric element and the sliver acoustic backing layer selected from the both side surfaces along the longitudinal direction of the sliver laminate structure. In this stage, the signal side electrode is connected to the first electrode alone of the sliver piezoelectric element. Also, the ground side electrode is insulated from the first electrode of the sliver piezoelectric element with an insulating member so as to be connected to the second electrode alone of the sliver piezoelectric element. As a result, formed are the signal side electrode and the ground side electrode extending from the side surface of the sliver piezoelectric element so as to reach the sliver acoustic backing layer. By setting appropriately the lengths of the signal side electrode and the ground side electrode extending to reach the acoustic backing layer, the signal side electrode is divided for each channel after the channel dividing operation. On the other hand, the ground side electrode is left common even after the channel dividing operation. Incidentally, it is possible to form an electrode group divided for each channel by forming the ground side electrode by the method similar to that for forming the signal side electrode.

In the next step, a dicing process (cutting) is applied to the sliver laminate structure from the side of the sliver acoustic matching layer toward the sliver acoustic backing layer so as to divide the sliver acoustic matching layer and the sliver piezoelectric, thereby forming a plurality of channels each including a piezoelectric element and an acoustic matching layer. In general, each channel has a width of 100 to 300 μm. Also, the acoustic backing layer is cut at a depth of, for example, about 100 to 300 μm so as to form trenches, thereby dividing for each channel the signal side electrode extending to reach one side surface of the acoustic backing layer. It should be noted, however, that the ground side electrode extending to reach the other side surface is left common even after the channel-dividing operation.

In the next step, the signal side flexible printed wiring board provided with a signal line patterned at a arranging pitch of the piezoelectric elements is bonded to one side surface of the sliver acoustic backing layer so as to be connected to the divided signal side electrode. Then, the ground side flexible printed wiring board provided with a ground line that is made common is bonded to the other side surface of the sliver acoustic backing layer so as to be connected to the ground side electrode.

By the process described above, manufactured is a channel array unit of a one column arrangement, in which one column of channels, each including the piezoelectric element and the acoustic matching layer, are arranged on the acoustic backing layer at a prescribed pitch, and the signal side electrode and the ground side electrode are electrically connected to the signal line and the ground line, respectively, formed on the flexible printed wiring board on each side surface of the acoustic backing layer.

Further, a filler member made of, for example, a silicone resin is buried in the space between the adjacent channels, and an acoustic lens is bonded to each of the plural channels. Finally, the resultant structure is housed in a case loaded with a control circuit for controlling the drive timing of the piezoelectric element of each channel and with a signal processing circuit including an amplifying circuit for amplifying the signal received by the laminated piezoelectric element, thereby manufacturing a desired two dimensional array-type ultrasonic probe.

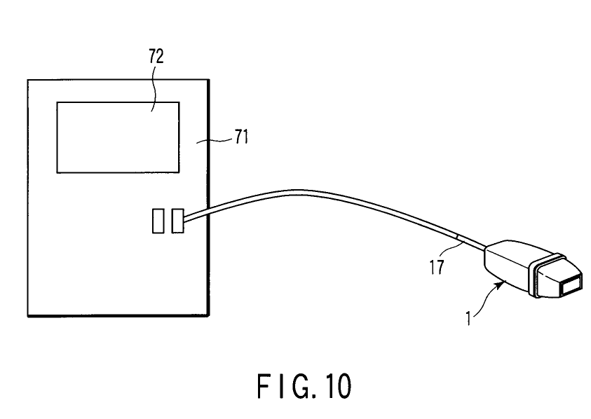

An ultrasonic diagnostic apparatus equipped with the ultrasonic probe according to an embodiment will now be described with reference to FIG. 10.

A medical ultrasonic diagnostic apparatus (or an ultrasonic image inspecting apparatus), which transmits ultrasonic signals to an object and receives the reflected signals (echo signals) coming from the object for forming an image of the object, comprises an array-type ultrasonic probe performing the function of transmitting and receiving ultrasonic signals. The ultrasonic probe is constructed as shown in, for example, FIGS. 1 to 3. As shown in FIG. 10, the ultrasonic probe 1 is connected to an ultrasonic diagnostic apparatus 71 via a cable 17. The ultrasonic diagnostic apparatus 71 comprises an ultrasonic probe controller (not shown) for transmitting ultrasonic signals from the ultrasonic probe to an object and receiving the ultrasonic signals reflected from the object. The ultrasonic diagnostic apparatus 71 also comprises a display 72.

The ultrasonic probe according to the embodiment described above comprises an acoustic backing layer exhibiting an acoustic impedance of 1.3 to 6 MRayls at a room temperature of 25° C. and made of a composite resin material including a resin and a plurality of bonded fibers contained in the resin, each of the bonded fibers being formed of a plurality of zinc oxide fibers bonded to each other at one edge portions and extending in different directions in the other edge portions. The ultrasonic probe also comprises a plurality of channels arranged on the backing layer with space, each channel including a acoustic matching layer formed on the piezoelectric element and an acoustic lens formed to cover at least the surface of the acoustic matching layer. The ultrasonic probe of the particular construction produces prominent effects as pointed out below:

(1) The acoustic backing layer is made of a composite resin material including a resin and a plurality of bonded fibers contained in the resin, each of the bonded fibers being formed of a plurality of zinc oxide fibers bonded to each other at one edge portions and extending in different directions in the other edge portions and, thus, is capable of exhibiting a large acoustic attenuation factor of the ultrasonic wave. To be more specific, the ultrasonic wave generated by the driving of the piezoelectric elements of the plural channels is released from the back side of the piezoelectric element so as to reach the bonded fibers contained in the acoustic backing layer. Since the bonded fiber is formed of a plurality of zinc oxide fibers bonded to each other at one portions and extending in different directions in the other edge portions, the bonded fiber itself is vibrated upon receipt of the ultrasonic wave so as to absorb the ultrasonic wave, thereby exhibiting a large acoustic attenuation factor.

(2) The zinc oxide fiber constituting the bonded fibers contained in the composite resin material exhibits an excellent thermal conductivity. Therefore, the heat generated by the piezoelectric elements of the plural channels and the heat generated in accordance with damping of the ultrasonic wave within the acoustic backing layer itself can be released effectively to the outside.

(3) The acoustic backing layer made of a composite resin material including a resin and a plurality of bonded fibers contained in the resin, each of the bonded fibers being formed of a plurality of zinc oxide fibers bonded to each other at one edge portions and extending in different directions in the other edge portions exhibits a high mechanical strength. Therefore, it is possible to prevent the acoustic backing layer from being cracked in the steps of cutting the acoustic matching layer and the piezoelectric element for forming the channels, forming the trenches in the acoustic backing layer, and cutting the acoustic matching layer, the piezoelectric element and the acoustic backing layer. It should also be noted that, in the acoustic backing layer of the particular construction, it is possible to permit the strengths in the thickness direction and in the planar direction to be balanced by the bonded fiber contained the acoustic backing layer so as to make it possible to moderate satisfactorily the stress in the cutting stage, thereby preventing the crack occurrence. As a result, it is possible to prevent the channel defect.

As pointed out above, the ultrasonic probe of the embodiment comprises an acoustic backing layer exhibiting a large acoustic attenuation factor and high thermal conductivity. In addition, since the channel defect can be prevented by the improvement in the strength of the acoustic backing layer, it is possible to provide an ultrasonic probe having a high sensitivity and a high reliability.

In the embodiment, an epoxy resin or a silicone based rubber is used as the resin for forming the composite resin material so as to make it possible to use an epoxy resin as an adhesive resin layer. It follows that it is possible to bond strongly the piezoelectric element and the acoustic backing layer to each other. It is also possible to prevent more effectively the channel defect.

Particularly, the bonded fibers contained in the acoustic backing layer are defined to be formed of a plurality of zinc oxide fibers, each having a diameter not larger than 10 μm and a length at least 5 times as much as the diameter. Also, the bonded fibers are defined to be contained in the resin in an amount of 3 to 20% by volume. This particular construction permits the acoustic backing layer of the ultrasonic probe to exhibit a higher strength, a higher acoustic attenuation factor and higher thermal conductivity so as to make it possible to provide an ultrasonic probe exhibiting a higher sensitivity and a higher reliability.

It is also possible for the composite resin material used for preparing the acoustic backing layer to be further loaded with particles of metal tungsten, tungsten oxide, metal tantalum, or iron oxide so as to make it possible to provide an ultrasonic probe equipped with an acoustic backing layer exhibiting a further improved strength, a high acoustic attenuation factor and high thermal conductivity and, thus, further improved in sensitivity and reliability.

The ultrasonic diagnostic apparatus according to the embodiment comprises an ultrasonic probe exhibiting a high sensitivity and a high reliability and, thus, permits improving the quality of the tomographic image and improving the sensitivity.

The present invention will now be described more in detail with reference to Examples.

### EXAMPLE 1

A plurality of tetrapod-like zinc oxide fibers (TPZ) forming bonded fibers were added to “Ecobond 27”, which is a trade name of a liquid epoxy resin manufactured by Emerson & Coming Inc., hereinafter abbreviated as “EPR”. The zinc oxide fibers (TPZ) were added to the liquid epoxy resin EPR in an amount of 3% by weight based on the sum of the liquid epoxy resin EPR and TPZ, followed by further adding a curing agent to the mixture. Then, the resultant composition was put in a polyethylene cup and stirred and uniformly mixed for 5 minutes by using a rotary mixer, thereby preparing an acoustic backing composition. The PZT used was formed of four zinc oxide fibers bonded to each other at one edge portions and extending in different directions in the other edge portions. Each of the zinc oxide fibers had a diameter of about 2 μm and a length of 40 to 50 μm. Also, the plural bonded fibers (TPZ) included bonded fibers consisting of four zinc oxide fibers having substantially the same lengths and extending in different directions and also included other bonded fibers consisting of four zinc oxide fibers having the lengths falling within a range of 40 to 50 μm and extending in different directions.

In the next step, the acoustic backing composition was defoamed for 10 minutes within a vacuum container and, then, put in a cup formed of Teflon (registered trademark). Further, the composition was subjected to a preliminary curing at 85° C. for one hour, followed by further curing the composition at 125° C. for 2 hours so as to manufacture the raw material of the acoustic backing layer.

### EXAMPLES 2 TO 7

The raw material of the acoustic backing layer was manufactured as in Example 1, except that EPR, TZP and a second filler were mixed in the mixing ratio shown in Table 1.

### EXAMPLE 8

A plurality of tetrapod-like zinc oxide fibers (TPZ) similar to those used in Example 1 were added to a liquid NBR (trade name of a liquid nitrile rubber manufactured by Nippon Zeon Inc., which is hereinafter abbreviated as NBR) in an amount of 5% by volume based on the sum of NBR and TPZ. Then, Fe₂O₃ particles having an average particle diameter of 5 to 10 μm were added to the mixture in an amount of 5% by volume based on the sum of NBR and the iron oxide particles. The resultant composition was put in a polyethylene cup and stirred for 5 minutes by using a rotary mixer so as to mix uniformly the composition, thereby obtaining an acoustic backing composition. The acoustic backing composition was defoamed for 10 minutes in a vacuum container, followed by putting the defoamed composition in a cup formed of Teflon (registered trademark). Further, the raw material of the acoustic backing layer was manufactured by curing the defoamed composition at 80° C. for 24 hours.

### EXAMPLE 9

A plurality of tetrapod-like zinc oxide fibers (TPZ) similar to those used in Example 1 were added to “Kurare LIR” (trade name of isoprene rubber manufactured Kurare Inc., which is hereinafter abbreviated as IR) in an amount of 10% by volume based on the sum of IR and TPZ. Then, tungsten particles having an average particle diameter of 5 to 10 μm were added to the mixture in an amount of 10% by volume based on the sum of IR and the tungsten particles. The resultant composition was put in a polyethylene cup and slowly stirred for 5 minutes by using a rotary mixer so as to mix uniformly the composition, thereby obtaining an acoustic backing composition. The acoustic backing composition was defoamed for 10 minutes in a vacuum container, followed by putting the defoamed composition in a cup formed of Teflon (registered trademark). Further, the raw material of the acoustic backing layer was manufactured by curing the defoamed composition at 80° C. for 24 hours.

### EXAMPLE 10

A plurality of tetrapod-like zinc oxide fibers (TPZ) similar to those used in Example 1 were added to “TSE 32221S” (trade name of a silicone rubber manufactured by Momentive Performance Materials Japan Co., which is hereinafter abbreviated as (Q) in an amount of 4% by volume based on the sum of Q and TPZ. Then, Fe₂O₃ particles having an average particle diameter of 5 to 10 μm were added to the mixture in an amount of 4% by volume based on the sum of Q and the iron oxide particles. The resultant composition was put in a polyethylene cup and stirred for 5 minutes by using a rotary mixer so as to mix uniformly the composition, thereby obtaining an acoustic backing composition. The acoustic backing composition was defoamed for 10 minutes in a vacuum container, followed by putting the defoamed composition in a cup formed of Teflon (registered trademark). Further, the raw material of the acoustic backing layer was manufactured by curing the defoamed composition at 40° C. for 24 hours.

### EXAMPLE 11

The raw material of the acoustic backing layer was manufactured as in Example 10, except that the mixing ratio of Q, TZP and the second filler was changed as shown in Table 1.

### COMPARATIVE EXAMPLES 1 AND 2

The raw material of the acoustic backing layer was manufactured as in Example 1, except that EPR and the first filler or EPR, the first filler and the second filler were mixed at the mixing ratio shown in Table 1.

### COMPARATIVE EXAMPLE 3

The raw material of the acoustic backing layer was manufactured as in Example 8, except that NBR, the first filler and the second filler were mixed at the mixing ratio shown in Table 1.

### COMPARATIVE EXAMPLE 4

The raw material of the acoustic backing layer was manufactured as in Example 9, except that IR, the first filler and the second filler were mixed at the mixing ratio shown in Table 1.

### COMPARATIVE EXAMPLES 5 AND 6

The raw material of the acoustic backing layer was manufactured as in Example 10, except that Q, the first filler and the second filler were mixed at the mixing ratio shown in Table 1.

Incidentally, the abbreviations CF, GF and AF given in the column of the first filler in Table 1 denotes the kind and the size of the fibers as follows:

CF: Carbon fibers having a diameter of 10 μm and a length of 6 mm;

GF: Glass fibers having a diameter of 10 μm and a length of 6 mm;

AF: Alumina fibers having a diameter of 10 μm and a length of 6 mm;

The second fillers used in all of the Examples and the Comparative Examples were formed of particles having average particle diameter of 5 to 10 μm. Also, the amount of the first filler shown in Table 1 denotes the percentage by volume based on the sum of the resin and the first filler, and the amount of the second filler denotes the percentage by volume based on the sum of the resin and the second filler.

The density, the acoustic velocity, the acoustic impedance (Z), the acoustic attenuation factor, the thermal conductivity, the insulation resistance, the signal nonuniformity and the dicing machinability were measured as follows in respect of the raw material of the acoustic backing layer obtained in each of Examples 1 to 11 an Comparative Examples 1 to 6:

1) Density:

The density was obtained by the Archimedean method by measuring the weight of a first test piece cut from the raw material of the acoustic backing layer and sized at 30 mm×30 mm×1 mm. The weight of the first test piece was measured in the air at 25° C. and within water.

2) Acoustic Velocity and Acoustic Attenuation Factor:

The acoustic velocity and the acoustic attenuation factor of the first test piece were measured in water at 25° C. by using a measuring probe of 1 MHz. To be more specific, ultrasonic signals were transmitted from an ultrasonic probe to a stainless steel plate and the first test piece arranged within water so as to measure the echo signals reflected from the first test piece.

The acoustic velocity was obtained from the difference in time between the echo signals caused by the presence of the first test piece and from the thickness of the first test piece. The acoustic velocity (C) was calculated by using the formula given below by utilizing the difference in time of the transmitted waveform between water and the first test piece, with the acoustic velocity of water at each temperature used as the standard:

C=C₀/[L−C₀(Δt/d)]

where C₀ denotes the acoustic velocity of water, L denotes the distance between the ultrasonic probe and the sample (object to be measured), d denotes the thickness of the sample, Δt denotes the difference in time of the zero-cross point after the transmitted waveforms of water and the first test piece passed through the first peak.

The acoustic attenuation factor was obtained by a prescribed method from the difference in intensity in the water at 25° C. between the reflected echo signals that was caused by the presence of the first test piece and from the thickness of the first test piece.

3) Acoustic Impedance (Z):

The acoustic impedance (Z) was obtained as the product between the density and the acoustic velocity measured by the methods described above.

4) Thermal Conductivity:

The thermal conductivity was obtained by the laser flush method using a second test piece cut from the raw material of the acoustic backing layer and having a diameter of 10 mm and a thickness of 1 mm.

5) Insulation Resistance:

The insulation resistance was evaluated by applying a voltage of 500 V to the first test piece sized at 30 mm×30 mm×1 mm for one minute so as to measure the leaking current.

The insulation resistance was evaluated in five stages as given below:

5: The resistivity was not lower than 1×10¹² Ω·cm;

4: The resistivity was not lower than 1×10¹¹ Ω·cm and lower than 1×10¹² Ω·cm;

3: The resistivity was not lower than 1×10¹⁰ Ω·cm and lower than 1×10¹¹ Ω·cm;

2: The resistivity was not lower than 1×10⁹ Ω·cm and lower than 1×10¹⁰ Ω·cm;

1: The resistivity was lower than 1×10⁹ Ω·cm.

6) Signal Nonuniformity:

A PZT piezoelectric element and an acoustic matching layer formed of a mixed material prepared by adding alumina used as an inorganic filler to a silicone rubber were laminated one upon the other in the order mentioned on the first test piece sized at 30 mm×30 mm×1 mm, with an epoxy resin adhesive layer formed between the piezoelectric element and the acoustic matching layer, so as to form a laminate structure. Then, a cross dicing was applied from the acoustic matching layer toward the acoustic backing layer of the laminate structure so as to have trenches having a depth of 200 μm formed in the acoustic backing layer, thereby forming 400 channels in total (20 ×20) each having an area of 200 μm×200 μm (i.e., 0.04 mm²). A pulse voltage of 100 V was applied to one electrode of the piezoelectric element of each channel and the other electrode was connected to the ground so as to vibrate the piezoelectric element. The signal nonuniformity relating to the channel defect was evaluated by measuring the signal intensity when the piezoelectric element of each channel was vibrated. The signal nonuniformity was evaluated in five stages, as given below:

5: The nonuniformity of the signal intensity among the channels was not larger than 5%;

4: The nonuniformity of the signal intensity among the channels exceeded 5% but was not larger than 10%;

3: The nonuniformity of the signal intensity among the channels exceeded 10% but was not larger than 20%;

2: The nonuniformity of the signal intensity among the channels exceeded 20% but was not larger than 40%;

1: The nonuniformity of the signal intensity among the channels was not smaller than 40%.

7) Dicing Machinability:

A cross-dicing was applied to the first test piece sized at 30 mm×30 mm×1 mm so as to form trenches each having a depth of 200 μm in the surface region of the first test piece. Formed were 400 trenches in total (20×20) each sized at 200 μm×200 μm. The broken state of the trenches was observed with a microscope so as to evaluate the dicing machinability. The dicing machinability was evaluated in five stages as given below:

5: The number of broken trenches was 0%;

4: The number of broken trenches exceeded 0% but was not larger than 1%;

3: The number of broken trenches exceeded 1% but was not larger than 5%;

2: The number of broken trenches exceeded 5% but was not larger than 20%;

1: The number of broken trenches exceeded 20%.

Table 1 also shows the measured values of the density, the acoustic velocity, etc. of the raw material of the acoustic backing layer.

TABLE 1

| | | First | | Second | | | Acoustic | |
|---|---|---|---|---|---|---|---|---|
| | | filler | | filler | | Density | velocity | Z |
| | Resin | Kind | Amount | Kind | Amount | (g/cm³) | (m/s) | (MRayls) |
| Example 1 | EPR | TPZ | 3 | — | 0 | 1.24 | 2500 | 3.10 |
| Example 2 | EPR | TPZ | 10 | — | 0 | 1.57 | 2400 | 3.77 |
| Example 3 | EPR | TPZ | 20 | Fe₂O₃ | 10 | 2.45 | 2300 | 5.64 |
| Example 4 | EPR | TPZ | 4 | W | 4 | 1.97 | 2200 | 4.33 |
| Example 5 | EPR | TPZ | 5 | WO₂ | 5 | 1.83 | 2100 | 3.84 |
| Example 6 | EPR | TPZ | 6 | Ta | 6 | 2.26 | 2000 | 4.52 |
| Example 7 | EPR | TPZ | 40 | SiO₂ | 5 | 3.03 | 2250 | 6.82 |
| Example 8 | NBR | TPZ | 5 | Fe | 5 | 1.58 | 1440 | 2.28 |
| Example 9 | IR | TPZ | 10 | W | 10 | 3.24 | 1400 | 4.54 |
| Example 10 | Q | TPZ | 4 | Fe₂O₃ | 4 | 1.36 | 955 | 1.30 |
| Example 11 | Q | TPZ | 10 | WO₃ | 10 | 2.09 | 900 | 1.88 |
| Comparative | EPR | — | 0 | W | 1 | 2.90 | 2100 | 6.09 |
| Example 1 | | | | | | | | |
| Comparative | EPR | CF | 5 | W | 10 | 2.93 | 2200 | 6.45 |
| Example 2 | | | | | | | | |
| Comparative | NBR | GF | 10 | Al₂O₃ | 10 | 1.23 | 1500 | 1.85 |
| Example 3 | | | | | | | | |
| Comparative | IR | GF | 5 | Fe₂O₃ | 5 | 1.21 | 1450 | 1.75 |
| Example 4 | | | | | | | | |
| Comparative | Q | AF | 10 | SiO₂ | 10 | 1.41 | 950 | 1.34 |
| Example 5 | | | | | | | | |
| Comparative | Q | CF | 5 | Ag | 30 | 3.89 | 920 | 3.58 |
| Example 6 | | | | | | | | |

| | Acoustic | Thermal | | | |
|---|---|---|---|---|---|
| | attenuation factor | conductivity | Insulation | Signal | Dicing |
| | (dB/mm/MHz) | (W/mK) | resistance | nonuniformity | machinability |
| Example 1 | 1.5 | 0.5 | 5 | 5 | 4 |
| Example 2 | 2.4 | 0.7 | 4 | 5 | 4 |
| Example 3 | 4.2 | 1.5 | 4 | 5 | 5 |
| Example 4 | 3 | 0.6 | 5 | 5 | 4 |
| Example 5 | 2.55 | 0.7 | 5 | 5 | 4 |
| Example 6 | 3.9 | 0.8 | 4 | 5 | 4 |
| Example 7 | 4.2 | 2.8 | 2 | 5 | 2 |
| Example 8 | 5.6 | 0.7 | 4 | 5 | 4 |
| Example 9 | 4.5 | 1 | 4 | 5 | 5 |
| Example 10 | 2.1 | 0.65 | 5 | 5 | 4 |
| Example 11 | 2.5 | 1.2 | 5 | 5 | 5 |
| Comparative | 2.1 | 0.25 | 5 | 2 | 1 |
| Example 1 | | | | | |
| Comparative | 3.5 | 0.98 | 2 | 1 | 4 |
| Example 2 | | | | | |
| Comparative | 2.9 | 1.8 | 2 | 1 | 5 |
| Example 3 | | | | | |
| Comparative | 3.9 | 0.52 | 4 | 1 | 5 |
| Example 4 | | | | | |
| Comparative | 1.42 | 1.2 | 4 | 2 | 2 |
| Example 5 | | | | | |
| Comparative | 3.5 | 8.2 | 1 | 1 | 1 |
| Example 6 | | | | | |

As apparent from Table 1, the raw material of the acoustic backing layer for each of Examples 1 to 11, which was prepared from a composite resin material obtained by adding a plurality of tetrapod-like bonded fibers each formed of zinc oxide fibers (TPZ) to a resin, exhibited a large acoustic attenuation factor of 1.5 to 5.6 dB/mm MHz and a high thermal conductivity of 0.5 to 1.5 W/mK in spite of the small acoustic impedance of 1.5 to 6 MRayls. In addition, the raw material of the acoustic backing layer used in the present invention was found to be low in the defective ratio of the fine channel having an area of 0.04 mm² and to be small in the nonuniformity of the sensitivity.

Particularly, the raw material of the acoustic backing layer for each of Examples 1 to 10, which was obtained from a composite resin material containing 3 to 20% by volume of the bonded fibers formed of tetrapod-like zinc oxide fibers (PZT), was found to exhibit the insulation resistance and the dicing machinability higher than those of the raw material of the acoustic backing layer for Example 11, which was obtained from a composite resin material containing TPZ in an amount exceeding 20% by volume.

Incidentally, the Examples described above are simply intended to exemplify the apparatus for embodying the technical idea of the present invention. Needless to say, the technical idea of the present invention is not limited by the Examples described above in respect of the material, shape, construction, arrangement, etc. of the constituting members. In other words, the present invention can be worked in variously modified fashions within the technical scope of the present invention.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic probe, comprising:
an acoustic backing layer made of a composite resin material including a resin and a plurality of bonded fibers contained in the resin, each of the bonded fibers being formed of a plurality of zinc oxide fibers bonded to each other at one edge portions and extending in different directions in the other edge portions, the composite resin material exhibiting an acoustic impedance of 1.3 to 6 MRayls at 25° C.;
a plurality of channels arranged on the acoustic backing layer with space, each channel having a piezoelectric element and an acoustic matching layer formed on the piezoelectric element; and
an acoustic lens formed to cover at least the surface of the acoustic matching layer of each channel.

2. The ultrasonic probe according to claim 1, wherein the zinc oxide fiber has a diameter not larger than 10 μm and a length at least 5 times as much as the diameter, and the bonded fibers consisting of the zinc oxide fibers are contained in the resin in an amount of 3 to 20% by volume based on the sum of the resin and the bonded fibers.

3. The ultrasonic probe according to claim 1, wherein the upper limit of the length of the zinc oxide fiber is 50 μm.

4. The ultrasonic probe according to claim 1, wherein the bonded fibers are formed of four zinc oxide fibers bonded to each other at one edge portions and extending in different directions in the other edge portions.

5. The ultrasonic probe according to claim 1, wherein the resin is at least one material selected from the group consisting of an epoxy resin, a silicone based rubber, an isoprene rubber and a nitrile based rubber.

6. The ultrasonic probe according to claim 1, wherein the resin is selected from the group consisting of an epoxy resin and a silicone based rubber, and the acoustic backing layer made of the composite resin material containing the resin and the piezoelectric element of each of the plural channels arranged on the acoustic backing layer are bonded to each other with an epoxy resin adhesive.

7. The ultrasonic probe according to claim 1, wherein the composite resin material further contains at least one kind of particles made of material selected from the group consisting of metal tungsten, tungsten oxide, metal tantalum, metal iron and iron oxide.

8. The ultrasonic probe according to claim 7, wherein the particles have an average particle diameter of 2 to 15 μm.

9. The ultrasonic probe according to claim 7, wherein the particles are contained in the resin in an amount not larger than 10% by volume based on the sum of the resin and the particles.

10. The ultrasonic probe according to claim 7, wherein the bonded fibers and the particles are contained in the resin in a volume ratio of 3:0.1 to 2:1.

11. The ultrasonic probe according to claim 1, wherein trenches corresponding to the spaces between the adjacent channels are further formed in the acoustic backing layer.

12. The ultrasonic probe according to claim 1, wherein the plural channels are arranged in a two dimensional direction so as to form an array and each channel has an area not larger than 0.03 mm².

13. An ultrasonic diagnostic apparatus, comprising:
an ultrasonic probe comprising an acoustic backing layer made of a composite resin material including a resin and a plurality of bonded fibers contained in the resin, each of the bonded fibers being formed of a plurality of zinc oxide fibers bonded to each other at one edge portions and extending in different directions in the other edge portions, the composite resin material exhibiting an acoustic impedance of 1.3 to 6 MRayls at 25° C., a plurality of channels arranged on the acoustic backing layer with space, each channel having a piezoelectric element and an acoustic matching layer formed on the piezoelectric element, and an acoustic lens formed to cover at least the surface of the acoustic matching layer of each channel; and
an ultrasonic diagnostic apparatus member connected to the ultrasonic probe via a cable.
